(19) United States
(12) Patent Application Publication
(10) Pub. No.: US 2022/0211901 A1
(43) Pub. Date: Jul. 7, 2022

(54) THROMBIN-FREE HEMOSTATIC MATERIALS, METHODS OF MANUFACTURE, AND USES THEREOF

(71) Applicants: BAXTER INTERNATIONAL INC., Deerfield, IL (US); BAXTER HEALTHCARE SA, Glattpark (Opfikon) (CH)

(72) Inventor: Yves Delmotte, Boca Raton, FL (US)

(21) Appl. No.: 17/647,128

(22) Filed: Jan. 5, 2022

Related U.S. Application Data
(60) Provisional application No. 63/134,691, filed on Jan. 7, 2021.

(51) Int. Cl. A61L 24/10

(52) U.S. Cl. CPC A61L 24/106; A61L 24/108

(57) ABSTRACT

Disclosed herein are hemostatic materials and methods of production and use thereof.

### RELATED APPLICATIONS

This application claims the benefit of U.S. Provisional Patent Application No. 63/134,691, filed Jan. 7, 2021, the disclosure of which is incorporated herein by reference in its entirety.

### FIELD

The present disclosure relates to the production and use of hemostatic materials.

### BACKGROUND

The rapid control of topical bleeding is of critical importance in wound management, especially for the management of trauma, e.g., as a result of injury or surgery. Conventional methods of controlling bleeding at active bleeding sites employ the use of “passive” devices including cotton gauze pads. Passive devices, however, do not have the ability to initiate or accelerate blood clotting.

In contrast to passive devices, hemostats are “active” substances that promote hemostasis through the use of hemostatic agents, for example, fibrinogen or thrombin, and actively participate in the coagulation cascade to form a fibrin clot. As the key coagulation protease, thrombin converts soluble fibrinogen into fibrin networks by cleaving fibrinogen into fibrin monomers, which aggregate to form a three-dimensional hydrogel.

Fibrin “glue” is a formulation used to create a fibrin clot for hemostasis or wound healing. It is typically produced from co-packaged fibrinogen and thrombin. Current fibrin glues use a cocktail of proteins containing fibrinogen as a substrate and thrombin as its catalyst; a substance which helps increase the rate of chemical change and is recovered unchanged chemically at the end of chemical reaction.

With some current fibrin glues, fibrinogen and thrombin are respectively reconstituted with water/aprotinin and calcium chloride solution. Equal volumes of the fibrinogen and thrombin solutions are drawn into separate syringes to be simultaneously administrated. This is an example of homogeneous catalysis, wherein the catalyst is present in the same phase as the reactant(s). However, fibrinogen solutions can have a relatively high viscosity (>90 cps), while the viscosity of thrombin is much lower (similar to that of water). Mixing these two components effectively can be challenging, particularly at low flow rates such as those generated within a syringe. Therefore, improved systems, devices, and methods are desirable.

### SUMMARY

The instant disclosure provides a novel class of hemostats, and methods of manufacturing thereof, for use in methods for establishing local hemostasis. Disclosed embodiments provide an essentially thrombin-free fibrin composition, and methods of manufacture and use thereof, said methods comprising heterogeneous catalysis.

Use of heterogeneous instead of homogeneous catalysis enables the use of a single syringe or container, and eliminates the complications associated with managing parameters such as viscosity and back pressure. Through the use of disclosed embodiments, mixing of fibrinogen and thrombin is no longer required, thus simplifying the administration of a fibrin glue such as TISSEEL® to a single syringe. Further, viscosity is no longer problematic, as fibrinogen can be directly diluted to obtain 50 mg/ml formulations. Because the solution viscosity is low, fibrin produced with heterogeneous catalysis can be easily sprayed and delivered, for example through a catheter. For example, in embodiments, the viscosity of the produced fibrin composition can be, for example, 90 cps, 80 cps, 70 cps, 60 cps, 50 cps, 40 cps, 30 cps, 20 cps, 10 cps, 5 cps, 1 cps, or the like.

In embodiments, adsorption of thrombin can be performed on the inner wall of ancillaries, for example devices and equipment such as tubes, pipes, vessels, and the like, that can be connected to a syringe, for example the luer of a syringe, containing the fibrinogen solution.

In embodiments, the need for calcium chloride in forming a fibrin clot is eliminated.

Embodiments disclosed herein comprise methods of adsorbing thrombin on to a surface.

Embodiments disclosed herein comprise methods of producing an essentially thrombin-free fibrin using heterogeneous catalysis.

Embodiments disclosed herein comprise compositions comprising an essentially thrombin-free fibrin.

Embodiments disclosed herein comprise compositions comprising an essentially thrombin-free fibrin made by a process comprising heterogeneous catalysis.

Embodiments disclosed herein comprise thrombin formulations that are stable at room-temperature (RT). For example, in current formulations, thrombin in solution is not stable at RT because it is autocatalytic, degrading itself in a concentration-dependent manner. Hence, TISSEEL® must be used after thawing at RT within 48 to 72 hours, ARTISS® within 1-2 weeks and VISTASEAL™ within 24 hours. The surface-adsorbed thrombin of the current disclosure can be stored in a dry state which increases its stability.

Disclosed embodiments comprise methods of use comprising an essentially thrombin-free fibrin. For example, disclosed methods and devices can be used to reduce or stop bleeding, for example bleeding associated with surgical procedures, injuries, wounds, and the like. Embodiments can comprise treatment of various classes of bleeding, including:

Class I involves up to 15% of blood volume. There is typically no change in vital signs and fluid resuscitation is not usually necessary.

Class II involves 15-30% of total blood volume. A patient is often tachycardic (rapid heart beat) with a reduction in the difference between the systolic and diastolic blood pressures.

Class III involves loss of 30-40% of circulating blood volume. The patient's blood pressure drops, the heart rate increases, peripheral hypo-perfusion (shock) with diminished capillary refill occurs, and the mental status worsens.

Class IV involves loss of >40% of circulating blood volume. The limit of the body's compensation ability is reached and aggressive resuscitation is required to prevent death.

### BRIEF DESCRIPTION OF THE DRAWINGS

FIG. 1 shows neutralization of thrombin adsorbed on glass beads and in solution (control). The left hand graph shows that heparin with or without anti-thrombin (AT) does not neutralize the thrombin adsorbed on the glass beads. The right hand graph shows that the thrombin in solution is neutralized by the complex AT heparin.

FIG. 2 shows neutralization of thrombin adsorbed on glass beads and in solution (control). The left hand graph shows that thrombin in solution is neutralized by hirudin. The right hand graph shows that hirudin will also neutralize thrombin adsorbed on the glass beads, but at much higher concentration.

FIG. 3 shows endothelial cell capillary formation in fibrin. The left image shows the cell capillaries in fibrin obtained by using the thrombin adsorbed on glass beads (heterogeneously catalyzed). The right image shows cell capillaries in fibrin obtained with homogeneously catalyzed thrombin.

FIG. 4 shows a syringe connected with a glass tube; the interior of the glass tube serves as the solid support for adsorbing thrombin.

FIG. 5 shows fibrin “spaghetti” formed with a thrombin-adsorbed glass tube.

FIG. 6 shows that Fg+ does not polymerize regular fibrinogen solution (Fg). Fibrin (Fg+) generated with a thrombin coated glass tube was transferred in a vessel containing fibrinogen solution. The absence of thrombin is confirmed by the lack of clot formation of the fibrinogen solution.

FIG. 7 shows cannula with a VYON-F disk coated with thrombin.

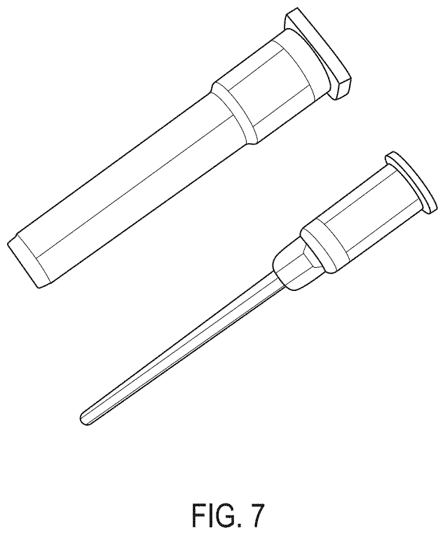

### DETAILED DESCRIPTION

Research efforts related to hemostatic materials have focused on the use of bioactive agents, specifically hemostatic agents. However, current formulations and devices do not adequately meet the needs of patients and practitioners. For example, certain current fibrin formulations such as glues require time-consuming preparation, and provide compositions that contain thrombin. In contrast, the instant disclosure provides methods for faster and more efficient preparation of fibrin hemostat formulations, including thrombin-free fibrin formulations.

### Definitions

“Administration,” or “to administer” means the step of giving (i.e. administering) a hemostatic system, device, material agent, or combination thereof to a subject. The materials disclosed herein can be administered via a number of appropriate routes.

“Entirely free” (“consisting of” terminology) means that within the detection range of the instrument or process being used, the substance cannot be detected or its presence cannot be confirmed.

“Essentially free” means that only small amounts of the substance can be detected

“Hemostatic agent” means an agent that can initiate and stabilize blood clot growth during bleeding, including biologics such as fibrin, thrombin, small molecules such as tranexamic acid (TXA), peptides such as Thrombin Receptor Activating Peptides (TRAPs), and inorganic materials such as kaolin.

“Patient” means a human or non-human subject receiving medical or veterinary care.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic result, such as by healing of injured or damaged tissue.

Thrombin Adsorption

Disclosed embodiments comprise methods for adsorbing thrombin on to a solid material, for example for use as a catalyst in the production of fibrin via a heterogeneous catalysis process. For example, disclosed embodiments comprise methods of adsorbing thrombin on to, for example, hydroxyapatite, glass, or the like. In embodiments, the solid material such as glass can comprise sheets, tubes, cylinders, beads, syringes, tubing, or the like. Suitable material can comprise porous or non-porous materials, or substantially non-porous materials, including, for example, plastics, metals, silicon, combinations thereof, and the like.

Disclosed methods comprise incubating, for example at room temperature (RT) a thrombin-containing solution with, for example, a solid material such as glass beads. In disclosed embodiments the incubation temperature can be, for example above RT or below RT, such as, for example 0° C., 5° C., 10° C., 15° C., 20° C., 25° C., 30° C., 35° C., 40° C., 45° C., 50° C., 55° C., or the like.

Disclosed embodiments comprise incubation of thrombin using solutions of various concentrations. For example, the thrombin solution can be at a concentration of, for example, 50 IU/ml, 100 IU/ml, 200 IU/ml, 300 IU/ml, 400 IU/ml, 500 IU/ml, 600 IU/ml, 700 IU/ml, 1000 IU/ml, 2000 IU/ml, 3000 IU/ml, 4000 IU/ml, 5000 IU/ml, 6000 IU/ml, 7000 IU/ml, 8000 IU/ml, 9000 IU/ml, 10,000 IU/ml, or more, or the like.

In embodiments, the quantity of solid material used for adsorbing can depend upon the amount of thrombin present in the reaction. For example, 100 mg of glass beads can be incubated with 50 μl of thrombin at 500 IU/ml and 50 μl of thrombin at 4 IU/ml for 2 hours at RT. In embodiments, incubation time can vary, for example in embodiments the suitable incubation time can be 0.5 hour, 1 hour, 2 hours, 3 hours, 4 hours, 5 hours, 6 hours, or more.

After incubation, the solid material can be washed to remove un-adsorbed thrombin.

Production of Thrombin-Free Fibrin

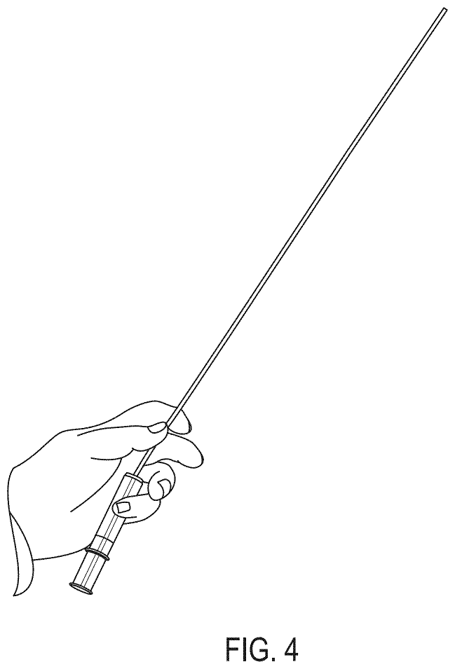

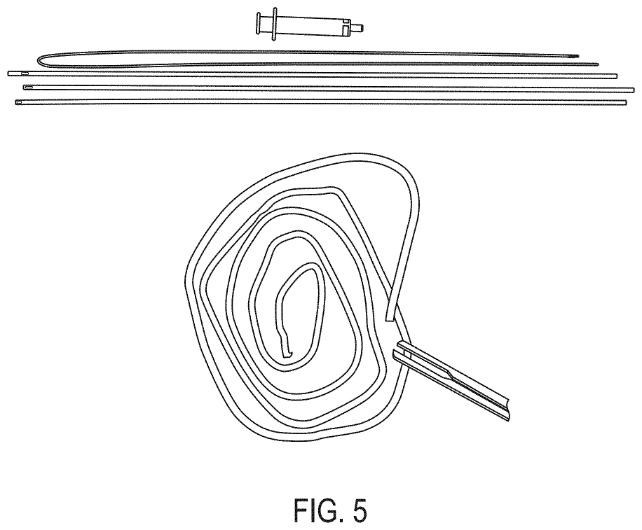

Disclosed embodiments comprise methods for producing fibrin, for example thrombin-free fibrin. In embodiments, a solid substrate comprising adsorbed thrombin, for example thrombin adsorbed to the surface of the solid substrate, is contacted with a fibrinogen solution. As the fibrinogen contacts the thrombin, the fibrinogen is cleaved to form a fibrin composition. For example, a fibrinogen solution can be passed through a glass tube (such as in FIG. 4) comprising thrombin adsorbed to the tube's interior surface. The fibrinogen is cleaved, and fibrin (for example in clotted form) exits the tube (as seen in FIG. 5).

Thrombin-Free Fibrin Compositions

Disclosed embodiments comprise hemostatic compositions, for example hemostatic fibrin compositions. In embodiments, the fibrin composition is essentially thrombin-free. In embodiments, the fibrin composition comprises, for example, less than 10% thrombin, less than 9% thrombin, less than 8% thrombin, less than 7% thrombin, less than 6% thrombin, less than 5% thrombin, less than 4% thrombin, less than 3% thrombin, less than 2% thrombin, less than 1% thrombin, less than 0.5% thrombin, less than 0.4% thrombin, less than 0.3% thrombin, less than 0.2% thrombin, less than 0.1% thrombin, less than 0.05% thrombin, less than 0.01% thrombin, less than 0.001% thrombin, or less, or the like.

In embodiments, a liquid fibrin composition as disclosed herein comprises, for example, less than 5 IU/mL thrombin, less than 4 IU/mL thrombin, less than 3 IU/mL thrombin, less than 2 IU/mL thrombin, less than 1 IU/mL thrombin, less than 0.5 IU/mL thrombin, less than 0.25 IU/mL thrombin, less than 0.125 IU/mL thrombin, less than 0.0625 IU/mL thrombin, or the like.

In embodiments, the fibrin composition is CaCl free.

Disclosed compositions can be stably stored at RT for several months. For example, TISSEEL® must be used after thawing at RT within 48 to 72 hours, ARTISS® within 1-2 weeks and VISTASEAL™ within 24 hours. The surface-adsorbed thrombin of the current disclosure can be stored in dry state which will considerably increase its stability. This improved stability allows storing a ready-to-use formulation, for example in an operating room or trauma center. Time-consuming preparation of kit components or long thawing times of a frozen product are thus avoided. For example, frozen products when thawed and warmed to 37° C. or thawed at RT must be used within 4 hours, or, these products are wasted. In contrast, disclosed embodiments can be ready for application in less than a minute to be, avoiding waste of human plasma-derived products.

Disclosed embodiments can comprise thrombin that is stable at RT, for example stable at RT for 1 week, 2 weeks, 3 weeks, 4 weeks, 5 weeks, 6 weeks, 7 weeks, 8 weeks, 12 weeks, 16 weeks, 20 weeks, or longer.

Commercial Products/Kits

The present hemostatic material can be finished as a commercial product by the usual steps performed in the present field, for example by appropriate sterilization and packaging steps. For example, the present material may be treated by UV/vis irradiation (200-500 nm), for example using photo-initiators with different absorption wavelengths (e.g. Irgacure 184, 2959), preferably water-soluble initiators (Irgacure 2959). Such irradiation is usually performed for an irradiation time of 1-60 min, but longer irradiation times may be applied, depending on the specific method. In embodiments, gamma or beta irradiation can be used, for example to sterilize the thrombin-coated substrate. For liquid components of the kit, solvent/detergent (SD) treatment or nanofiltration can be used for sterilization.

The material according to the present disclosure can be finally sterile-wrapped so as to retain sterility until use and packaged (e.g. by the addition of specific product information leaflets) into suitable containers (boxes, etc.).

Disclosed embodiments can also be provided in kit form combined with other components necessary for administration of the material to the patient. The kit may further contain means for administering or preparing for administering the hemostatic material, such as syringes, tubes, catheters, forceps, scissors, sterilizing pads or lotions, etc.

Disclosed kits, such as for use in surgery and/or in the treatment of injuries and/or wounds, can comprise a disclosed hemostatic material and at least one administration device, for example a buffer, a syringe, a tube, a catheter, forceps, scissors, gauze, a sterilizing pad or lotion.

In embodiments, the buffer solution further comprises an anti-bacterial agent, immunosuppressive agent, anti-inflammatory agent, anti-fibrinolytic agent, especially aprotinin or ECEA, growth factor, vitamin, cell, or mixtures thereof. Alternatively, the kit can also further comprise an anti-bacterial agent, immunosuppressive agent, anti-inflammatory agent, anti-fibrinolytic agent, especially aprotinin or ECEA, growth factor, vitamin, cell, or mixtures thereof. In embodiments, the buffer solution can comprise a salt, such as calcium chloride.

The kits are designed in various forms based on the specific deficiencies they are designed to treat.

Methods of Use

Methods of use of disclosed embodiments can comprise application to a site where bleeding is desired to be reduced, such as a site of injury or surgical procedure.

These methods are further described in the following Examples.

### EXAMPLES

The following non-limiting Examples are provided for illustrative purposes only in order to facilitate a more complete understanding of representative embodiments. These examples should not be construed to limit any of the embodiments described in the present specification.

### Example 1

### Adsorption of Thrombin

Different solid materials were preliminarily screened, such as Hillex micro carrier beads (Sigma M-4060), cytodex microcarrier beads (Sigma C-3150 et Aldrich C-0646-56), hydroxyapatite (M BCP Biomatlante lot 900) and different glass beads having an average size ranging from 45 μm to 300 μm (Sigma G-1277, G-4649, G-1145).

Further tests were then performed on glass beads (106 μm; Sigma G-4649).

Adsorption of Thrombin

100 mg of glass beads were respectively incubated with 50 μl of thrombin solution at 500 IU/ml and 50 μl at 4 IU/ml (from TISSEEL® and ARTISS® kits) for 2H at RT. After incubation, the beads were washed 6 times with 5 ml of PBS buffer, supernatants were discarded, and the final pellet was suspended in 200 μL of PBS buffer.

Quantity of Thrombin Adsorbed

A chromo-thrombin kit was used for determination of the amount of thrombin adsorbed on the material. The amount of thrombin bound to beads was determined by measuring at 405 nm the liberation of para-nitroanilin (yellow color) from a synthetic substrate.

100 μl of chromo-thrombin was added to 100 μl of the pellet suspension. A calibration curve was performed with thrombin at different concentrations:

1 IU/ml;

0.5 IU/ml;

0.25 IU/ml;

0.12 IU/ml;

0.06 IU/ml;

0.03 IU/ml; and

0.015 IU/ml.

Optical density (OD) was measured at 405 nm over time. OD of the sample was corrected by taking into account the OD of an equivalent volume of untreated glass beads/chromo-thrombin and the OD of 100 μl of the last washing solution added with 100 μl of chromo-thrombin.

Results:

0.065 IU thrombin/ml of suspension for the beads treated with 500 IU/ml thrombin

<0.01 IU thrombin/ml of suspension for the beads treated with 4 IU/ml thrombin.

The solution of thrombin used contained a significant amount of albumin that will compete with thrombin during the adsorption process. For further tests, a high concentration of pure thrombin is recommended.

Tests done with the same amount of hydroxyapatite granule from Biomatlante showed that the quantity of thrombin adsorbed was 10 to 25 times higher than with glass, this can be explained by the micro and macro porosity of the granule.

### Example 2

### Determination of Clotting Time

100 mg of dried thrombin adsorbed pellets were covered with 300 μl of fibrinogen at 5 mg/ml without agitation. A homogeneous fibrin clot was observed after 13 min.

100 mg of dried thrombin adsorbed pellets were covered with 5 ml of fibrinogen at 5 mg/ml without agitation. A homogeneous fibrin clot was observed after 2 hours.

A) Heparin solutions at 0.5 u/ml, 0.25 u/ml. 0.12 u/ml, and 0.06 u/ml were prepared

The following samples were prepared in tubes.

Heparin was complemented with anti-thrombin (AT) or PBS buffer:

| | | | | | | | | | | |
|---|---|---|---|---|---|---|---|---|---|---|
| Heparin 100 μl | 0.5 | u/ml | 0.25 | u/ml | 0.12 | u/ml | 0.06 | u/ml | PBS 100 | μl |
| With AT or PBS | 50 | μl | 50 | μl | 50 | μl | 50 | μl | 50 | μl |
| Pellet Supernatant | 100 | μl | 100 | μl | 100 | μl | 100 | μl | 100 | μl |
| 0.06 IU/ml | | | | | | | | | | |
| Chromothrombin | 100 | μl | 100 | μl | 100 | μl | 100 | μl | 100 | μl |

Samples were incubated at RT, and tubes were then centrifuged.

100 μl of supernatant were used for measurement of para-nitroanilin release at 405 nm.

Control: a thrombin solution at a 0.06 IU/ml was processed as the pellet.

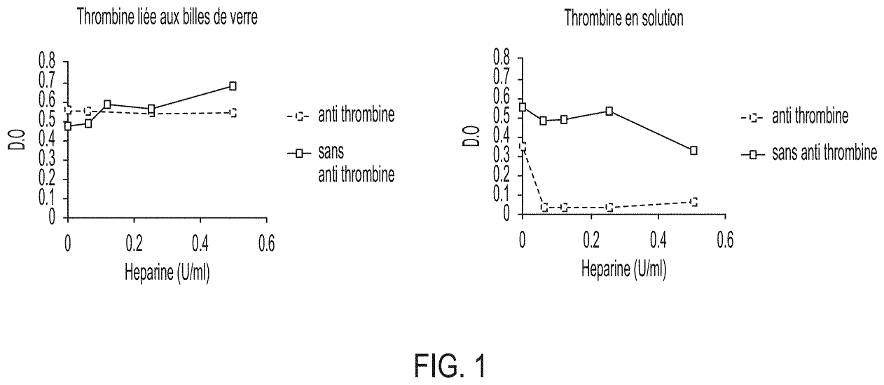

FIG. 1 shows the results of this experiment. The left hand graph shows that heparin with or W/O AT does not neutralize the thrombin adsorbed on the glass beads.

Right hand graph shows as expected that the thrombin in solution is neutralized by the complexed AT heparin.

B) Hirudin solutions at 100 ng/ml, 50 ng/ml, 25 ng/ml were prepared in tubes as shown in the table below.

| | | | | | | | | |
|---|---|---|---|---|---|---|---|---|
| Hirudin 50 μl | 100 | ng/ml | 50 | ng/ml | 25 | ng/ml | PBS 50 | μl |
| Pellet Supernatant | 50 | μl | 50 | μl | 50 | μl | 50 | μl |
| Chromothrombin | 50 | μl | 50 | μl | 50 | μl | 50 | μl |

Samples were incubated at RT.

Tubes were centrifuged

100 μl of supernatant were used for measurement of para-nitroanilin release at 405 nm.

Control: a thrombin solution at a 0.07 IU/ml was processed as the pellet, but using lower concentration of hirudin.

| | | | | | | | | | | | | |
|---|---|---|---|---|---|---|---|---|---|---|---|---|
| Hirudin 50 μl | 2.5 | ng/ml | 1.2 | ng/ml | 0.6 | ng/ml | 0.3 | ng/ml | 0.15 | ng/ml | PBS 50 | μl |
| Thrombin | 50 | μl | 50 | μl | 50 | μl | 50 | μl | 50 | μl | 50 | μl |
| 0.07 IU/ml | | | | | | | | | | | | |
| Chromothrombin | 50 | μl | 50 | μl | 50 | μl | 50 | μl | 50 | μl | 50 | μl |

Samples were incubated at RT.

100 μl of supernatant were used for measurement of para-nitroanilin release at 405 nm.

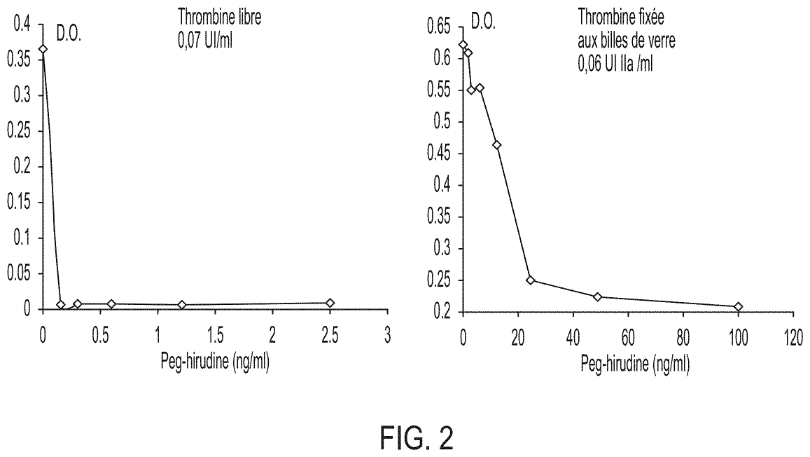

FIG. 2 shows the results of this experiment. The left hand graph shows that thrombin in solution is neutralized by hirudin. The right hand graph shows that hirudin does neutralize thrombin adsorbed on the glass beads, but at much higher concentration.

### Example 3

### Angiogenesis in Fibrin Formed with Thrombin Adsorbed on Glass

It is known that culture of endothelial cells in a 3D network of fibrin leads to the formation of capillary tubes. However, the formation of the capillaries depends on the quality and the structure of the fibrin network. Here, we tested if fibrin formed by the coagulation of fibrinogen with thrombin adsorbed on glass beads produces organization of endothelial cells in capillary tubes (angiogenesis).

Using a 96 well plate, 10,000 endothelial cells were added in each well and allowed to attach. Supernatant was removed, and 50 μg of fibrinogen at 10 mg/ml was added to each well. 10 ul of pellet supernatant (test item) or 10 μl of thrombin at a concentration equivalent to the adsorbed thrombin was then added to each well as Control. Coagulation was performed at RT.

150 μl of culture medium MEM containing 10% calf serum, 5% glutamine, and the necessary factors for endothelial growth were added to each well.

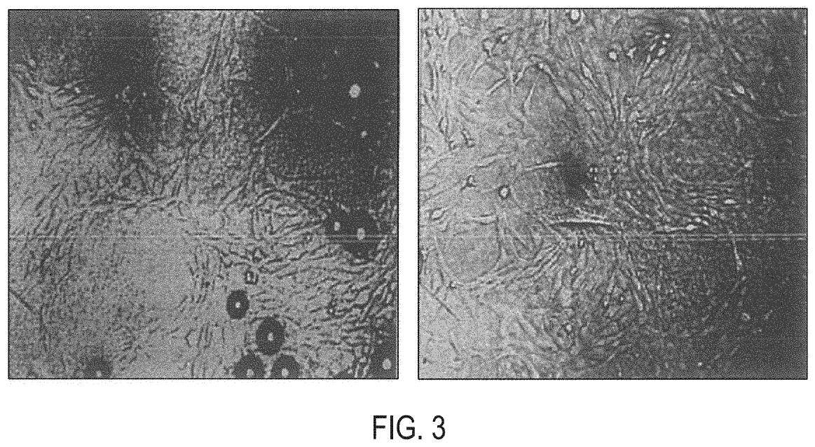

Incubation was performed over 3 days, then capillary tubes were observed and captured using an image analysis system. FIG. 3 shows the formation of capillaries in fibrin. The left hand picture shows the capillaries in fibrin obtained by using the thrombin adsorbed on glass beads. The right hand picture shows the fibrin obtained with the regular thrombin.

### Example 4

### Thrombin Homogeneity

A glass tube (glasroehren OD: 3 mm, ID: 1.6 mm, L: 500 mm. from Duran VWR 201-1003) was coated with thrombin at 500 IU/ml (Tissucol DUO lot: VND1N090 from Baxter). The glass tube was cleaned with ethanol to remove any material which could affect the adsorption of thrombin.

A syringe containing thrombin at 500 IU/ml was connected to the glass tube. Thrombin was injected from the bottom up until the glass tube was filled, and kept vertical for 30 min. The syringe was disconnected. Any excess of thrombin in the tube was flushed with 1 L of demineralized water.

A syringe containing fibrinogen at 45 mg/ml (dilution 1:2 of the fibrinogen from the Tissucol DUO lot: VND1N090 kit) was connected to the tube. The plunger was actuated to push the fibrinogen solution up to the top of the tube held vertically.

FIG. 4 shows the system consisting of the syringe and the internally thrombin coated glass tube. Polymerization occurred after 30 min

FIG. 5 shows three glass tubes that were used for polymerizing fibrinogen.

Fibrin formed in one tube was extruded as shown on the lower picture. Another test that was done to assess the homogeneity and the mechanical property of the fibrin spaghetti formed is an elongation test.

The upper image of FIG. 5 is fibrin “spaghetti” elongated up to 100%. It formed a U shape tube at twice the length of the glass tube initially hosting it. This simple test demonstrates that in the absence of calcium, homogeneous and well-structured fibrin can be obtained when using heterogeneous catalysis.

Test 2: Fibrin obtained via heterogeneous catalysis is thrombin-free. The same experiment as in Test 1 was performed except that we did not wait until polymerization will be achieved in the glass tube. Fibrinogen was flushed through the thrombin coated tube and poured on a solution of fibrinogen preliminary placed into a plastic cuvette. Fibrinogen that flushed through the thrombin coated tube was labeled Fg+, this material has been exposed to thrombin and starts to polymerize over time. The purpose of this experiment was to check if thrombin could desorb from the inner wall surface of the tube when fibrinogen flushed the tube

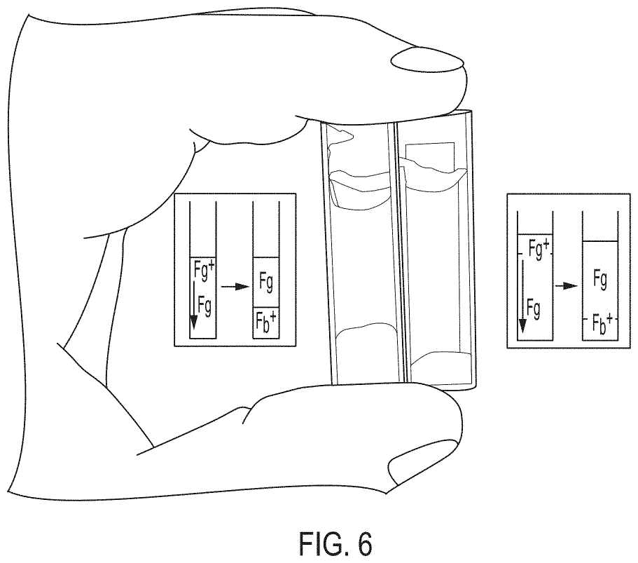

FIG. 6 illustrates the experiment. The left cuvette was filled with 2 ml of fibrinogen at 50 mg/ml and 1 ml of Fg+ is poured on top. The image on the left shows that FG+ is moving down while regular fibrinogen (Fg) is moving up due to the different densities. No polymerization of fibrinogen was taking place over the entire observation period of 5 days indicating that Fg+ is thrombin free.

The right cuvette was filled with 3 ml of fibrinogen at 50 mg/ml and 0.5 ml of Fg+ was poured on the top. The image on the right of FIG. 6 shows that Fg+ is moving down while regular fibrinogen (Fg) is moving up due to the different densities. No polymerization of fibrinogen was taking place over the entire observation period of 5 days indicating that Fg+ is thrombin free. Fibrinogen remained liquid and has been easily removed.

This test demonstrated that thrombin-free fibrin can be obtained when using the heterogeneous catalysis instead of homogeneous catalysis.

### Example 5

### Treatment of Injury

An automobile accident victim sustains traumatic injuries to the abdomen. To stop blood loss, a disclosed thrombin-free hemostat is applied to the injury site. Blood loss is reduced within minutes.

### Example 6

### Treatment of Injury

An automobile accident victim sustains traumatic injuries to the legs. To stop blood loss, a disclosed thrombin-free hemostat is applied to the injury site. Blood loss is reduced within minutes.

### Example 7

### Treatment of Injury

An automobile accident victim sustains traumatic injuries to the torso. To stop blood loss, a disclosed thrombin-free hemostat is applied to the injury site. Blood loss is reduced within minutes.

### Example 8

### Treatment of Wound

A Marine sustains traumatic gunshot injuries. To stop blood loss, a disclosed thrombin-free hemostat is applied to the injury site. Blood loss is reduced within minutes.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A method of preparing a liquid fibrin composition, said method comprising passing a fibrinogen solution over a solid phase substrate, said substrate comprising thrombin.

2. The method of claim 1, wherein said liquid fibrin composition comprises less than 2 IU thrombin per mL.

3. The method of claim 1, wherein said liquid fibrin composition comprises less than 1 IU thrombin per mL.

4. The method of claim 1, wherein said liquid fibrin composition comprises less than 0.5 IU thrombin per mL.

5. The method of claim 1, wherein said liquid fibrin composition comprises less than 0.25 IU thrombin per mL.

6. The method of claim 1, wherein said liquid fibrin composition comprises less than 0.125 IU thrombin per mL.

7. A liquid hemostatic material comprising fibrin, wherein said composition comprises less than 2 IU thrombin per mL.

8. The liquid hemostatic material of claim 7, wherein said composition comprises less than 1 IU thrombin per mL.

9. The liquid hemostatic material of claim 8, wherein said composition comprises less than 0.5 IU thrombin per mL.

10. The liquid hemostatic material of claim 9, wherein said composition comprises less than 0.25 IU thrombin per mL.

11. The liquid hemostatic material of claim 10, wherein said composition comprises less than 0.125 IU thrombin per mL.

12. The liquid hemostatic material of claim 11, wherein said composition comprises less than 0.0625 IU thrombin per mL.

13. A hemostatic material comprising fibrin, made by a process comprising heterogeneous catalysis.

14. The hemostatic material of claim 13, wherein said heterogeneous catalysis comprises a liquid phase substrate and a solid phase catalyst.

15. The hemostatic material of claim 14, wherein said liquid phase substrate comprises fibrinogen.

16. The hemostatic material of claim 15, wherein said solid phase catalyst comprises thrombin.

17. A kit for use in establishing local hemostasis, comprising;
a fibrinogen composition;
a solid substrate comprising thrombin adsorbed to the surface of the substrate;
and at least one administration device, buffer solution, syringe, tube, catheter, forceps, scissors, sterilizing pad or lotion.
